(19) United States
(12) Patent Application Publication
(10) Pub. No.: US 2021/0322227 A1
(43) Pub. Date: Oct. 21, 2021

(54) ANTIMICROBIAL FOAM ARTICLES AND METHOD OF MAKING THE SAME

(71) Applicant: 3M INNOVATIVE PROPERTIES COMPANY, St. Paul, MN (US)

(72) Inventors: Petra L. Kohler Riedi, Minneapolis, MN (US); Joseph D. Rule, Woodbury, MN (US); Naimul Karim, Maplewood, MN (US); Semra Colak Atan, Saint Louis Park, MN (US); Wei Zhang, Woodbury, MN (US); Andrew W. Vail, Woodbury, MN (US)

(21) Appl. No.: 17/260,616

(22) PCT Filed: Jul. 19, 2019
(86) PCT No.: PCT/IB2019/056210

§ 371 Date: Jan. 15, 2021

Related U.S. Application Data
(60) Provisional application No. 62/712,039, filed on Jul. 30, 2018.

(51) Int. Cl. A61F 13/00; A61K 9/70; C08L 75/04

(52) U.S. Cl. CPC A61F 13/00063; A61F 13/00017; A61F 13/00021; C08L 2203/14; A61K 9/7092; C08L 75/04; A61F 13/00034

(57) ABSTRACT

An article. The article includes a polymer foam having a first major surface and a second major surface; and discrete domains of a therapeutic composition at least partially surrounded by the polymer foam; wherein an exterior surface of each discrete domain substantially conforms to a portion of the first major surface of the polymer foam; wherein the article comprises a first major surface and a second major surface; and wherein a portion of the first major surface of the polymer foam and the exterior surfaces of discrete domains form the first major surface of the article.

### BACKGROUND

Chronic wounds are often stalled by microbial infections. In these infections, microorganisms can colonize the wound and treatment with antimicrobial wound care products can be helpful to move chronic wounds into a trajectory of healing. Often these microorganisms grow in complex communities called biofilms. Bioflilms occur in over 80% of chronic wounds and are widely recognized to stall wound healing.

In treating complex, chronic wounds, clinicians often need to use products which can manage wound exudate. Absorbent foam wound dressings are widely used for exudate management and antimicrobial foam wound dressings are commercially available. Many commercially available wound dressings contain antimicrobial ingredients which are impregnated throughout the foam. There is a desire to a better article to deliver a greater amount of active material directly to the wound bed.

### SUMMARY

In one aspect, the present disclosure provides an article, comprising: a polymer foam having a first major surface and a second major surface; and discrete domains of a therapeutic composition at least partially surrounded by the polymer foam; wherein an exterior surface of each discrete domain substantially conforms to a portion of the first major surface of the polymer foam; wherein the article comprises a first major surface and a second major surface; and wherein a portion of the first major surface of the polymer foam and the exterior surfaces of discrete domains form the first major surface of the article.

In another aspect, the present disclosure provides a method of making a wound dressing, comprising: depositing a therapeutic composition onto a surface of a liner to form a printed surface; and disposing a foam material on the printed surface.

In another aspect, the present disclosure provides a method of making a wound dressing, comprising: depositing a first therapeutic composition onto a surface of a first liner to form a first printed surface; depositing a second therapeutic composition onto a surface of a second liner to form a second printed surface; and disposing a foam material between the first printed surface and the second printed surface.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### Definitions

For the following defined terms, these definitions shall be applied for the entire Specification, including the claims, unless a different definition is provided in the claims or elsewhere in the Specification based upon a specific reference to a modification of a term used in the following definitions:

The terms “about” or “approximately” with reference to a numerical value or a shape means +/−five percent of the numerical value or property or characteristic, but also expressly includes any narrow range within the +/−five percent of the numerical value or property or characteristic as well as the exact numerical value. For example, a temperature of “about” 100° C. refers to a temperature from 95° C. to 105° C., but also expressly includes any narrower range of temperature or even a single temperature within that range, including, for example, a temperature of exactly 100° C. For example, a viscosity of “about” 1 Pa-sec refers to a viscosity from 0.95 to 1.05 Pa-sec, but also expressly includes a viscosity of exactly 1 Pa-sec. Similarly, a perimeter that is “substantially square” is intended to describe a geometric shape having four lateral edges in which each lateral edge has a length which is from 95% to 105% of the length of any other lateral edge, but which also includes a geometric shape in which each lateral edge has exactly the same length.

The term “substantially” with reference to a property or characteristic means that the property or characteristic is exhibited to a greater extent than the opposite of that property or characteristic is exhibited. For example, a substrate that is “substantially” transparent refers to a substrate that transmits more radiation (e.g. visible light) than it fails to transmit (e.g. absorbs and reflects). Thus, a substrate that transmits more than 50% of the visible light incident upon its surface is substantially transparent, but a substrate that transmits 50% or less of the visible light incident upon its surface is not substantially transparent.

The terms “a”, “an”, and “the” include plural referents unless the content clearly dictates otherwise. Thus, for example, reference to a material containing “a compound” includes a mixture of two or more compounds.

### BRIEF DESCRIPTION OF THE DRAWINGS

The disclosure may be more completely understood in consideration of the following detailed description of various embodiments of the disclosure in connection with the accompanying figures, in which:

FIG. 1 is a cross-section view of one embodiment of the article.

FIG. 2A is a photograph of a cross section of the article.

FIG. 2B is a photograph of a top view of the article.

While the above-identified drawings, which may not be drawn to scale, set forth various embodiments of the present disclosure, other embodiments are also contemplated, as noted in the Detailed Description. In all cases, this disclosure describes the presently disclosed invention by way of representation of exemplary embodiments and not by express limitations. It should be understood that numerous other modifications and embodiments can be devised by those skilled in the art, which fall within the scope and spirit of this disclosure.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

As used in this Specification, the recitation of numerical ranges by endpoints includes all numbers subsumed within that range (e.g. 1 to 5 includes 1, 1.5, 2, 2.75, 3, 3.8, 4, and 5, and the like).

Unless otherwise indicated, all numbers expressing quantities or ingredients, measurement of properties and so forth used in the Specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Many commercially available wound dressings contain antimicrobial ingredients which are impregnated throughout the foam. This format makes it difficult to deliver an effective amount of antimicrobial molecules to the wound bed where bacterial colonization occurs. One way to deliver a greater amount of active material directly to the wound bed is to provide a bolus of antimicrobial material on one surface of a foam wound dressing. However, coating a large amount of material onto one side of the wound dressing could potentially block the foam's ability to absorb moisture through the coated surface. The current application provides an article allowing for a large amount of material to be incorporated into the foam without creating topographical features on the foam.

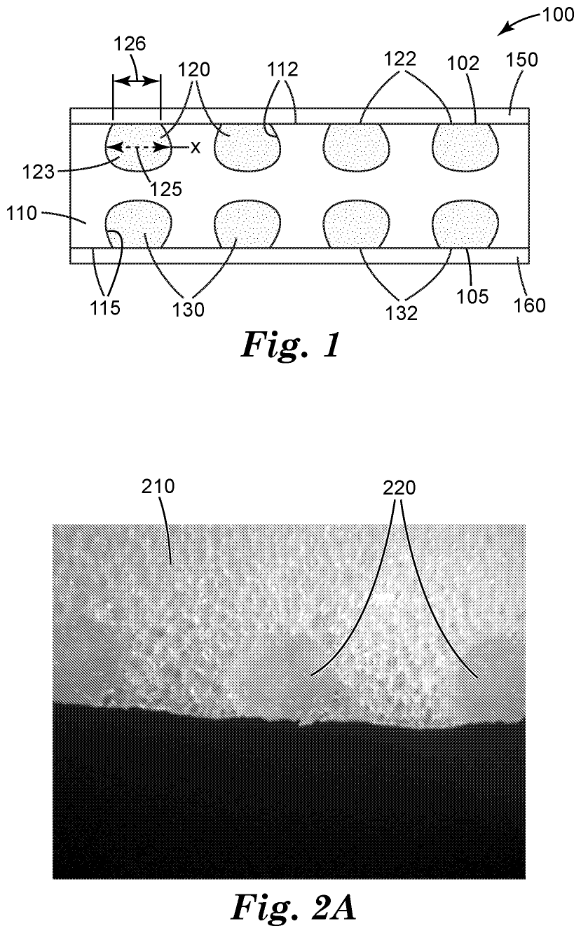

FIG. 1 is a schematic side view of one embodiment of article 100. The article 100 can include a polymer foam 110 having a first major surface 112 and a second major surface 115 and discrete domains 120 of a therapeutic composition. Discrete domains 120 can be at least partially surrounded by the polymer foam 110 as shown in FIG. 1. An exterior surface 122 of each discrete domain can substantially conform to (in other words, be coplanar with) a portion of the first major surface 112 of the polymer foam, as shown in FIG. 1. The article 100 can have a first major surface 102 and a second major surface 105. A portion of the first major surface 112 of the polymer foam 110 and the exterior surfaces 122 of discrete domains 120 form the first major surface 102 of the article 100. The exterior surfaces 122 of discrete domains 120 can cover between 1% and 99%, between 5% and 95%, between 10% and 90%, between 15% and 85%, between 20% and 80%, between 25% and 75%, or between 30% and 70% of the first major surface 102 of the article 100. The article 100 can further include a liner 150 on top of or adjacent to the first major surface 102 of the article 100.

In some embodiments, the article 100 can include additional discrete domains 130 of the therapeutic composition at least partially surrounded by the polymer foam 110. In these embodiments, the exterior surface 132 of each additional discrete domain 130 substantially conforms to a portion of the second major surface 115 of the polymer foam 110. A portion of the second major surface 115 of the polymer foam 110 and the exterior surfaces 132 of additional discrete domains 130 form the second major surface 105 of the article 100. The exterior surfaces 132 of discrete domains 130 can cover between 1% and 99%, between 5% and 95%, between 10% and 90%, between 15% and 85%, between 20% and 80%, between 25% and 75%, or between 30% and 70% of the second major surface 105 of the article 100. The article 100 can further include a second liner 160 on top of or adjacent to the second major surface 105 of the article 100.

In some embodiments, at least at one plane that is parallel to the first major surface 102 of the article 100 and between the first and the second major surface of the article, the cross section of each discrete domain is greater than the cross section of the exterior surface of the discrete domain. For example, at x plane as shown in FIG. 1, the cross section 125 of discrete domain 123 is greater than the cross section 126 of the exterior surface of the discrete domain 123. This can, for example, allow for enhanced mechanical locking of the therapeutic composition domains within the foam. In some embodiments, the therapeutic composition can be retained in the discrete domains.

In some embodiments, discrete domains can form a pattern. The pattern can be continuous or discontinuous. In some embodiments of continuous patterns, a continuous nature of discrete domains is less likely to fracture, and additionally, the shape of the pattern can be more precisely controlled because it does not require wicking within the foam, which can be complex and difficult to control. In some embodiments, discrete domains can form a predetermined pattern such that they are equally spaced from one another and possess a generally uniform size and shape. In some embodiments, discrete domains can form a random pattern such that they may be differently spaced from one another and may possess a generally different size and shape. In some embodiments, discrete domains can be sprinkled across the article. In some embodiments, the diameter of discrete domains can be at least about 100 microns, at least about 200 microns, at least about 300 microns, at least about 400 microns, at least about 500 microns, at least about 600 microns, at least about 700 microns, at least about 800 microns, at least about 900 microns or at least about 1000 microns. In some embodiments, the diameter of discrete domains can be less than 10 cm. In some embodiments, the diameter of discrete domains can be in a range from 100 microns to 10 cm. In some embodiments, discrete domains may have irregularly formed perimeters. This can mean that the discrete domains Slave irregular shapes (that is, no lines of symmetry). They may have edges that are not smooth (e.g., jagged or feathery edges). Irregularly formed discrete domains can also have a variety of thicknesses of the polymer foam surrounding the discrete domains.

In some embodiments, the discrete domains are shaped as channels that are at least partially surrounded by the foam. In some embodiments, the channels have the cross sectional shape of a half-circle or half-ellipse.

In some embodiments, the discrete domains are shaped as truncated spheres or truncated spheroids. In some embodiments, the discrete domains can be in a striped pattern. The striped pattern can be linear or curved, for example sinusoidal pattern. In some embodiments, the striped pattern can be oriented in a parallel or non-parallel fashion.

In some embodiments, each discrete domain has a portion of the perimeter surface that is curved and a portion of the perimeter surface that is flat. At least a portion of the perimeter surface that is flat is exposed on the surface of the article.

In some embodiments, a cross section of each discrete domain has a portion of the perimeter that is curved and a portion of the perimeter that is linear. At least a portion of the perimeter that is linear is exposed on the surface of the article.

In some embodiments, a portion of each discrete domain is embedded in the foam and each discrete domain has an exposed surface that is at least partially surrounded by the first major surface of the foam. In some embodiments, the liner is in contact with the exposed surfaces of the discrete domains and is also in contact with the first major surface of the foam. In some embodiments, the liner has a slit dividing it into two sections.

Articles according to the present disclosure are useful. For example, they may be applied to skin and used as wound dressings (e.g., including occlusive dressings and pressure dressings), and transdermal parches. In the case of wound dressings, articles according to the present disclosure can allow for a high loading of active material into the foam wound dressing while maintaining a high surface area of the foam wound dressing that is in contact with the wound for exudate management. In some embodiments, the article of the present disclosure can be absorbent foam wound dressings.

The method of making the wound dressing article of the present disclosure can include depositing a therapeutic composition onto a surface of a liner to form a printed surface and disposing a foam material on the printed surface.

Alternatively, the method of making the wound dressing article of the present disclosure can include depositing a first therapeutic composition onto a surface of a first liner to form a first printed surface: depositing a second therapeutic composition onto a surface of a second liner to form a second printed surface and disposing a foam material between the first printed surface and the second printed surface. The first therapeutic composition may be same as or different from the second therapeutic composition.

In some embodiments, once deposited, the therapeutic composition forms discrete domains of the therapeutic composition on the surface of the liner. The foam material can be disposed over the discrete domains of the therapeutic composition. The therapeutic composition may be deposited onto the liner according to any pattern or image. Patterns can include any suitable patient, for example, combinations and arrays comprising dots, squares, diamonds, lines, circles, hexagons, triangles, and combinations thereof.

The method can further include curing the foam material to form a foam on the printed surface at temperatures ranging from about room temperature to about 300° F. Lower temperatures may be required to ensure stability of therapeutic composition. With lower temperatures, the curing time is longer. It will take about 10 to about 15 minutes for curing. At higher temperatures, when some therapeutic composition remain stable, the curing time is, for example, about 4-5 minutes.

In some embodiments, the liner can be optionally removed from the foam after curing step. When the liner is removed from the foam, the discrete domains of therapeutic composition are then embedded in the foam, creating an article with a flat surface. This method allows for a large amount of therapeutic composition to be incorporated into the foam without creating topographical features on the foam. The high profile, topographical features of the previous foam products make it difficult to handle the foam product.

In some embodiments, the therapeutic composition may be deposited by non-contact printing methods such as spraying or jetting methods. In other embodiments, the therapeutic composition may be disposited by a solvent coating process or hot melt coating. Useful contact printing methods include, for example, flexography, roll coating, knife-coating, doctor-blade coating, and gravure roll coating.

The polymer foam material used in the current disclosure may be selected from hydrophilic or hydrophobic polymers, depending upon the nature of the therapeutic composition Typical hydrophilic polymer foam materials may be selected from the group consisting of polyurethane, polyvinylacetate, polyvinyl alcohol (PVA), polyethylene, and medical grade silicone.

In some embodiments, the therapeutic composition can comprise an active agent. Exemplary useful active agents (e.g., therapeutics) include; herbal medicines, anti-inflammatory drugs, both steroidal (e.g., prednisolone, triamcinolone) and nonsteroidal (e.g., naproxen, piroxicam); antibacterials (e.g., penicillins, cephalosporins, erythromycin, tetracycline, gentamycin, sulfathiazole, nitrofurantoin, trimethoprim, and quinolones (e.g., norfloxacin, flumequine, and ibafloxacin), bacitracin and its salts, neomycin and its salts, polymixin B) and antiseptics (e.g., chlorhexidine, chlorhexidine gluconate, alexidine, octenidine, antimicrobial quaternary ammonium surfactants such as benzalkonium chloride, cetylpyridinium chloride, and cetyltrimethylammonium halides, phenols, cresols, triclosan, antibacterial natural oils, iodophors, quaternary ammonium compounds, protonated tertiary and secondary amine compounds, certain metal ions (e.g., silver, and copper) and their salts (e.g., silver gluconate, silver lactate, and silver sulfate), biguanides, triclosan, and polymeric antibacterials (e.g., polyhexamethylene biguanide, polymers having protonated primary, secondary and/or tertiary amines, and poly-quaternary amines), antimicrobial lipids such as those described in U.S. Publ. Pat. Appln. No. 2005/0089539 A1 (Scholz et al.), which include the C₈-C₁₂ alkyl monoesters and monoethers of glycerin and propylene glycol); antiprotozoals (e.g., metronidazole); cardiovascular pharmaceuticals (e.g., amlodipine besylate, nitroglycerin, nifedipine, losartan potassium, irbesartan, diltiazem hydrochloride, clopidogrel bisulfate, digoxin, abcixmiab, furosemide, amiodarone hydrochloride, beraprost, theophylline, pirbuterol, salmeterol, isoproterenol, and tocopheryl nicotinate); calcium channel blockers (e.g., nifedipine, diltiazem); enzyme inhibitors such as collagenase inhibitors, protease inhibitors, elastase inhibitors, lipoxygenase inhibitors (e.g., A64077), and angiotensin converting enzyme inhibitors (e.g., captopril, lisinopril); other antihypertensives (e.g., propranolol), leukotriene antagonists (e.g., ICI204, 219) antiulceratives such as H2 antagonists; steroidal hormones (e.g., progesterone, testosterone, estradiol, levonorgestrel, mycophenolate mofetil, and methylprednisolone); antivirals and/or immunomodulators (e.g., 1-isobutyl-1H-imidazo[4,5-c]quinolin-4-amine, 1-(2-hydroxyl-2-methylpropyl)-1H-imidazo[4,5-c]quinoline-4-amine, acyclovir); cardiotonics (e.g., digitalis, digoxin); antitussives (e.g., codeine, dextromethorphan), antihistamines (e.g., diphenhydramine, chlorpheniramine, terfenadine); exfoliants (e.g., alpha-hydroxy acids or beta-hydroxy acids); analgesics (e.g., tramadol hydrochloride, fentanyl, metamizole, ketoprofen, morphine sulfate, lysine acetylsalicylate, acetaminophen, ketorolac tromethamine, morphine, loxoprofen sodium, and ibuprofen); dermatological products (e.g., isotretinoin and clindamycin phosphate), anesthetics (e.g., propofol, midazolam hydrochloride, and lidocaine hydrochloride); migraine therapies (e.g., ergotamine, melatonin, sumatriptan, zolmitriptan, and rizatriptan); sedatives and hypnotics (e.g., zolpidem, zolpidem tartrate, triazolam, and hycosine butylbromide); imaging components (e.g., isohexyl, technetium, TC99M sestamibi, iomeprol, gadodiamide, ioversol, and iopromide); peptide hormones (e.g., human or animal growth hormones LHRH); cardioactive products such as atriopeptides; proteinaceous products (e.g., insulin); enzymes (e.g., anti-plague enzymes, lysozyme, dextranase), antinauseants (e.g., scopolamine); anticonvulsants (e.g., carbamazepine); immunosuppressives (e.g., cyclosporine); psychotherapeutics (e.g., diazepam, sertraline hydrochloride, venlafaxine, bupropion hydrochloride, olanzapine, buspirone hydrochloride, alprazolam, methylphenidate hydrochloride, fluvoxamine maleate, and ergoloid mesylates); sedatives (e.g., phenobarbital); and coagulants (e.g., heparin); analgesics (e.g., acetaminophen); antimigraine agents (e.g., ergotamine, melatonin, sumatriptan), cholesterol reducers (e.g., atorvastatin calcium, lovastatin, bezafibrate, ciprofibrate, and gemfibrozil); antiarrhythmic agents (e.g., flecamide), antiemetics (e.g., metoclopramide, ondansetron); blood modifiers (e.g., epoetin alfa, enoxaparin sodium, and antihemophilic factor); antiarthritic components (e.g., celecoxib, nabumetone, misoprostol, and rofecoxib); AIDS and AIDS-related pharmaceuticals (e.g., lamivudine, indinavir sulfate, and stavudine); diabetes and diabetes-related therapies (e.g., metformin hydrochloride, insulin, troglitazone, and acarbose); biologicals (e.g., hepatitis B vaccine, and hepatitis A vaccine); immune response modifiers (e.g., purine derivatives, adenine derivatives, and CpGs); anticancer agents (e.g., methotrexate, paclitaxel, carboplatin, tamoxifen citrate, docetaxel, epirubicin hydrochloride, leuprolide acetate, bicalutamide, goserelin acetate implant, irinotecan hydrochloride, gemcitabine hydrochloride, and sargramostim); gastrointestinal products (e.g., lansoprazole, ranitidine hydrochloride, famotidine, ondansetron hydrochloride, granisetron hydrochloride, sulfasalazine, and infliximab); respirators' therapies (e.g. loratadine, fexofenadine hydrochloride, cetirizine hydrochloride, fluticasone propionate, salmeterol xinafoate, and budesonide); immunosuppressives (e.g., cyclosporine); neurologic agents such as anxiolytic drugs; haemostatics; antiobesity agents; nicotine; algicides; and pharmaceutically acceptable salts and esters of the foregoing.

In some embodiments, the therapeutic composition can include an active agent selected from the group consisting of antimicrobial agents, antibiotics, antioxidants, platelet-derived growth factor, vitamin A, vitamin C, vitamin E, corticosteroids, silver sulphadiazme, polymixin B sulphate, fusidic acids, pirfenedine, interferon, therapeutic oils, plant extracts, animal extracts, pharmaceuticals, vitamins, hormones, antioxidants, emu oil, aloe vera, lavender oil, rosehip oil silver sulphadiazme, polymixin B, fusidic acic and pirfenedine.

Active agents may be selected from those that are nonreactive with the foam material and/or other components of the active composition, although reactive active agents may be used. Antimicrobial agents are typically selected based on the type of microorganisms the articles will encounter in a particular use. Exemplary antimicrobial agents can include penicillins, cephalosporins, erythromycin, tetracycline, gentamycin, sulfathiazole, nitrofurantoin, trimethoprim, quinolones (e.g., norfloxacin, flumequine, and ibafloxacin), bacitracin and its salts, neomycin and its salts, polymixin B) and antiseptics (e.g., chlorhexidine, chlorhexidine gluconate, alexidine, octenidine, antimicrobial quaternary ammonium surfactants such as benzalkonium chloride, cetylpyridinium chloride, and cetyltrimethylammonium halides, phenols, cresols, triclosan, antibacterial natural oils, iodophors, quaternary ammonium compounds, protonated tertiary and secondary amine compounds, certain metal ions (e.g., silver, and copper) and their salts (e.g., silver gluconate, silver lactate, and silver sulfate), biguanides, triclosan, and polymeric antibacterials (e.g., polyhexamethylene biguanide, polymers having protonated primary, secondary and/or tertiary amines, and polyquaternary amines), and antimicrobial lipids such as those described in U.S. Publ. Pat. Appln. No, 2005/0089539 A1 (Scholz et ah), which include the C₈-C₁₂ alkyl monoesters and monoethers of glycerin and propylene glycol, and microbe inhibiting materials including chelators (e.g. EDTA and organic acids). The active agent may be included in the therapeutic composition in any amount. In some embodiments, the active agent may be present in an amount of at least 10, 20, 30, 40, or even at least 50 percent by weight, based on the total, weight of the therapeutic composition.

In some embodiments, the therapeutic composition can comprise a carrier. The carrier may comprise a liquid (aqueous, solvent based or based on a blend of a solvent and water), a gel (a hydrogel), a cream, a paste, or a solid. In some embodiments, the carrier may be a liquid at about room temperature. In other embodiments, the carrier may be a solid at about room temperature. In some embodiments, the carrier may be a liquid at about the temperature of the oral cavity of a human, i.e., at about 37° C. In other embodiments, the carrier may be a solid at about the temperature of the oral cavity of a human. Exemplary liquid carriers include water, alcohols (e.g., ethanol), glycerol, sorbitol, and liquid silicones. Exemplary solid carriers include crystalline or waxy materials, for example, polyethylene glycol.

Each non-carrier component of the therapeutic composition may independently be dissolved, dispersed, suspended, or emulsified in the carrier. In some embodiments, at least one component of the therapeutic composition is dissolved in the carrier. In some embodiments, at least one component of the therapeutic composition is dispersed in the carrier. In some embodiments, at least one component of the therapeutic composition is suspended in the carrier. In some embodiments, at least one component of the therapeutic composition is emulsified in the carrier. In some embodiments, the active agent can be dissolved or dispersed in an aqueous liquid. In some embodiments, fee active agent can be dissolved or dispersed in a gel (a hydrogel), a cream, or a paste. In some embodiments, fee active agent can be dissolved or dispersed in a thixotropic gel.

In some embodiments, the therapeutic composition can comprises a rheology modifier. Rheology modifier can be any suitable rheology modifier, for example an organic or inorganic material; a soluble or a swellable polymer; a linear, branched or crosslinked polymer; a natural or synthetic polymer, etc. Rheology modifier can be used to make the rheology of the therapeutic composition suitable for the desired method of depositing the composition on a liner. In some embodiments. the rheology modifier can also affect the release kinetics of the active agents contained in the therapeutic composition.

The therapeutic composition comprises a carrier. The carrier may comprise a liquid, a solid, or both. In some embodiments, the carrier may be a liquid at about room temperature. In other embodiments, the carrier may be a solid at about room temperature. In some embodiments, the carrier may be a liquid at about the temperature of the oral cavity of a human, i.e., at about 37° C. In other embodiments, the carrier may be a solid at about the temperature of the oral cavity of a human. Exemplary liquid carriers include water, alcohols (e.g., ethanol), glycerol, sorbitol, and liquid silicones. Exemplary solid carriers include polymers such as natural rubber, butyl rubber, poly(isobutylene), elastomers, styrene-butadiene rubber, polysaccharides, and waxes (e.g., beeswax).

Each non-carrier component of the therapeutic composition may independently be dissolved, dispersed, suspended, or emulsified in the carrier. In some embodiments, at least one component of the therapeutic composition is dissolved in the carrier. In some embodiments, at least one component of the therapeutic composition is dispersed in the carrier. In some embodiments, at least one component of the therapeutic composition is suspended in the carrier. In some embodiments, at least one component of the therapeutic composition is emulsified in the carrier.

Liners feat are suitable for use in current disclosure can be made of kraft papers, polyethylene, polypropylene, polyester or composites of any of these materials. The liners can be coated wife release agents such as fluorochemicals or silicones. For example, U.S. Pat. No. 4,472,480, the disclosure of which is hereby incorporated by reference, describes low surface energy perfluorochemical liners. The preferred liners are papers, polyolefin films, or polyester films coated with silicone release materials. Examples of commercially available silicone coated release papers are POLYSLIK™ silicone release papers available from James River Co., H. P. Smith Division (Bedford Park, Ill.) and silicone release papers supplied by Daubert Chemical Co. (Dixon, Ill.). The most preferred liner is 1-60BKG-157 paper liner available from Daubert, which is a super calendared Kraft paper with a water-based silicone release surface. Alternatively fee wound dressing may be linerless and delivered in roll form such as described in U.S. Pat. No. 5,803,086.

The following embodiments are intended to be illustrative of the present disclosure and not limiting.

### Embodiments

Embodiment 1 is an article, comprising: a polymer foam having a first major surface and a second major surface; and discrete domains of a therapeutic composition at least partially surrounded by the polymer foam; wherein an exterior surface of each discrete domain substantially conforms to a portion of the first major surface of the polymer foam; wherein the article comprises a first major surface and a second major surface; and wherein a portion of the first major surface of the polymer foam and the exterior surfaces of discrete domains form the first major surface of the article.
Embodiment 2 is the article of embodiment 1, wherein the article is a wound dressing.
Embodiment 3 is the article of embodiments 1-2, wherein discrete domains form a pattern.
Embodiment 4 is the article of embodiments 1-3, wherein at least at one plane that is parallel to the first major surface of the article and between the first and the second major surface of the article, the cross section of each discrete domain is greater than the cross section of the exterior surface of the discrete domain.
Embodiment 5 is the article of embodiments 1-4, wherein the diameter of discrete domains is at least about 100 microns.
Embodiment 6 is the article of embodiments 1-5, wherein the exterior surfaces of discrete domains cover between 1% and 99% of the first major surface of the article.
Embodiment 7 is the article of embodiments 1-6, further comprising a liner on top of or adjacent to the first major surface of the article.
Embodiment 8 is the article of embodiments 1-7, further comprising additional discrete domains of the therapeutic composition at least partially surrounded by the polymer foam.
Embodiment 9 is the article of embodiment 8, wherein the exterior surface of each additional discrete domain substantially conforms to a portion of the second major surface of the polymer foam.
Embodiment 10 is the article of embodiment 8, wherein a portion of the second major surface of the polymer foam and the exterior surfaces of additional discrete domains form the second major surface of the article.
Embodiment 11 is the article of embodiments 1-10, further comprising a second liner on top of or adjacent to the second major surface of the article.
Embodiment 12 is the article of embodiments 1-11, wherein the polymer foam comprises materials selected from the group consisting of polyurethane, poly vinylacetate, polyvinylalcohol, polyethylene, and silicone.
Embodiment 13 is the article of embodiments 1-12, wherein the therapeutic composition is retained in the discrete domains.
Embodiment 14 is the article of embodiments 1-13, wherein the therapeutic composition comprises an active agent selected from the group consisting of antimicrobial agents, antibiotics, antioxidants, platelet-derived growth, factor, vitamin A, vitamin C, vitamin E, corticosteroids, silver sulphadiazine, polymixin B sulphate, fusidic acids, pirfenedine, interferon, therapeutic oils, plant extracts, animal extracts, pharmaceuticals, vitamins, hormones, antioxidants, emu oil, aloe vera, lavender oil, rosehip oil silver sulphadiazine, polymixin B, fusidic acic and pirfenedine.
Embodiment 15 is the article of embodiments 1-14, wherein the therapeutic composition comprises an antimicrobial agent.
Embodiment 16 is a method of making a wound dressing, comprising: depositing a therapeutic composition onto a surface of a liner to form a printed surface; and disposing a foam material on the printed surface.
Embodiment 17 is the method of embodiment 16, further comprising curing the foam material to form a foam on the printed surface.
Embodiment 18 is the method of embodiments 16-17, wherein depositing the therapeutic composition comprises depositing the therapeutic composition in a pattern onto the liner.
Embodiment 19 is the method of embodiments 16-18, further comprising removing the liner from the foam.
Embodiment 20 is a method of making a wound dressing, comprising: depositing a first therapeutic composition onto a surface of a first liner to form a first printed surface; depositing a second therapeutic composition onto a surface of a second liner to form a second printed surface; and disposing a foam material between the first printed surface and the second printed surface.
Embodiment 21 is an article comprising: a polymeric film liner; a polymer foam having a first major surface and a second major surface; and a plurality of discrete domains of a therapeutic composition; wherein a portion of each discrete domain is embedded in the foam and each discrete domain has an exposed surface that is at least partially surrounded by the first major surface of the foam; wherein the liner is in contact with the exposed surfaces of the discrete domains and is also in contact with the first major surface of the foam.
Embodiment 22 is the article of embodiment 21, wherein the liner has a slit dividing it into two sections.
Embodiment 23 is the article of any one of the embodiment 21-22, wherein the therapeutic composition comprises an antimicrobial agent.
Embodiment 24 is the article of embodiments 15 and 23, wherein the antimicrobial agent is selected from the group consisting of polyhexamethylene biguanide (PHMB), chlorhexidine, benzalkonium chloride, benzethonium chloride, silver salts, Neomycin, polymyxin B, bacitracin, and octenidine.
Embodiment 25 is the article of any one of the embodiments 15, 23 and 24, wherein the antimicrobial agent is dissolved or dispersed in a gel, a hydrogel, a cream, and a paste vehicle.
Embodiment 26 is the article of any one of the embodiments 1-15 and 21-25, wherein the article is an absorbent wound dressing.

The following working examples are intended to be illustrative of the present disclosure and not limiting.

### Examples

### Materials for Preparation of the Polymer Foam

SUPRASEC 9634 isocyanate, a modified methylene diphenyl diisocyanate (MDI), was obtained from Huntsman Chemical Company, The Woodlands, Tex. SUPRASEC 9634 isocyanate was reported to have the following properties: equivalent weight of 143 g/equivalent, functionality of 2.15, and isocyanate content of 29.3%.
CDB-33143 polyether polyol, prepared as a blend from glycerine, propylene oxide, and ethylene oxide, was obtained from the Carpenter Company, Richmond, Va. CDB-33143 polyether polyol was reported by the manufacturer to have the following properties: hydroxyl number of 142, functionality of 3, ethylene oxide content of 26%.
CARPOL GP-700 polyether polyol, prepared from glycerine and propylene oxide, was obtained from the Carpenter Company, Richmond, Va. CARPOL GP-700 polyether polyol was reported by the manufacturer to have the following properties: average hydroxyl number of 240, functionality of 3, ethylene oxide content of 0%.
ARCOL E-434 polyether polyol, a polyoxy-propylene triol modified with ethylene oxide, was obtained from the Bayer MaterialScience, Pittsburgh, VA. ARCOL E-434 polyether polyol was reported by the manufacturer to have the following properties: hydroxyl number of 33.8-37.2, ethylene oxide content of 15%.
Triethanolamine LFG (low freeze grade), 85% triethanolamine and 15% water, was obtained from the Quaker Chemical Corporation, Conshohocken, Pa.
DABCO 33-LV solution of triethylene diamine (33 weight percent) in dipropylene glycol was obtained from Air Products and Chemicals Incorporated, Allentown, Pa.
DABCO BL-17 tertiary amine catalyst was obtained from Air Products and Chemicals Incorporated.
DABCO DC-198 silicone glycol copolymer surfactant and DABCO BA-100 polymeric acid blocking agent were obtained from Air Products and Chemicals Incorporated.

### Materials for Preparation of the Antimicrobial Gel

COSMOCIL PG antimicrobial [polyhexamethylene biguanide (PHMB) as a 20% (w/w) solution in water] was obtained from Lonza Limited, Basel, Switzerland.
Poly(ethylene glycol) 4000 (PEG4000) was obtained from EMD Millipore, Billerica, Mass.
Polyglycerol-3 was obtained from Solvay S.A., Brussels, Belgium.
Propylene glycol monocaprylate (CAPMUL PG8) was obtained from the Abitec Corporation, Columbus, Ohio.
CAB-O-SIL M5 fumed silica was obtained from the Cabot Corporation, Boston, Mass.
PLANTAREN 810 UP alklyl polyglucoside surfactant was obtained from the BASF Personal Care Company, Florham Park, N.J.

### Preparation of the Antimicrobial Gel and Pattern Coated Release Liner

The total amount of each component used to prepare a batch of antimicrobial gel is reported in Table 1.

TABLE 1

| | Total Amount Per |
|---|---|
| Component | Batch (g) |
| Polyglycerol-3 | 326.0 |
| Poly(ethylene glycol) 4000 (PEG4000) | 95.0 |
| COSMOCIL PG antimicrobial [polyhexamethylene | 13.0 |
| biguanide (PHMB) 20% (w/w) in water] | |
| Propylene glycol monocaprylate (CAPMUL PG8) | 13.0 |
| PLANTAREN 810 UP alklyl polyglucoside surfactant | 0.8 |
| CAB-O-SIL M5 fumed silica | 53.0 |

The mixing vessel of a double planetary mixer (Charles Ross & Son Company, Hauppauge, N.Y.) was warmed to 70° C. with an external water bath. Polyglycerol-3 was added to the mixing vessel followed by PEG4000 and the mixture was blended at 25 rpm (revolutions per minute) for 30 minutes. Next, PHMB, propylene glycol monocaprylate (CAPMUL PG8), and PLANTAREN 810 UP alklyl polyglucoside surfactant were sequentially added to the mixing vessel and the mixture was blended at 35 rpm for 5 minutes. CAB-O-SIL M5 fumed silica was then added in three equal portions with 5 minutes of blending at 35 rpm following the addition of each portion. Unmixed material on the sides of the mixing vessel was scraped into the bulk mixture and the mixture was blended for an additional 10 minutes at 25 rpm. The resulting gel was removed from the mixing vessel and warmed in an oven at 65° C. for one hour. The warmed gel was loaded into a 20 mL syringe with a Luer slip tip. The gel was then dispensed as a pattern of non-intersecting lines (approximately 10 cm in length and spaced about 4 mm apart) on the release surface of a silicone-coated paper release liner. Each line was approximately 2 mm in diameter. The pattern was applied to a 10 cm by 3.5 cm section of the release liner.

### Example 1. Preparation of Foam Article

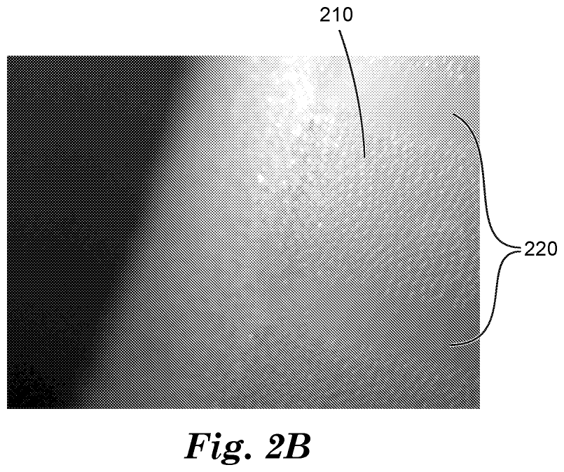

A layer of open cell polyurethane foam layer was cast by adding SUPRASEC 9634 isocyanate (58.2 parts) to a mixture of CDB-33143 polyether polyol (100 parts), CARPOL GP-700 polyether polyol (3.0 parts), water (1.0 parts), triethanolamine LFG (3.7 parts), DABCO DC-198 surfactant (2.0 parts), ARCOL E-434 polyether polyol (4.0 parts), DABCO 33-LV (0.45 parts), DABCO BA-100 polymeric acid blocking agent (0.12 parts), and DABCO BL-17 tertiary amine catalyst (0.10 parts). The formulation was scaled such that the total reaction mixture weighed 40 grams. The components were mixed in a plastic cup for 10 seconds at 3300 rpm using a DAC 150 FV speed mixer (Flacktek, Inc, Landrum S.C.). The entire mixture was then immediately poured over the gel pattern that had been previously applied to the silicone-coated paper release liner so that the entire gel pattern was covered by the mixture. The overall area of release liner covered by the foam was about 12 cm by 18 cm. A sheet of polypropylene coated release paper was placed over the exposed surface of the reactive foam mixture. The foam was allowed to rise and cure at room temperature. Image of a cross section of the foam article is shown as FIG. 2A and Image of a top view of the foam article is shown as FIG. 2B. As shown in FIG. 2A and FIG. 2B. Discrete domains 220 of antimicrobial gel was surrounded by polymer foam 210.

### Comparative Example A

The same procedure as reported in Example 1 was followed with the exception that after mixing, the foam formulation was immediately poured onto a silicone-coated release liner that did not have the applied gel pattern of Example 1.

### Example 2. Planktonic Microbial Kill Assay (Staphylococcus aureus)

A culture of Staphylococcus aureus strain number 6538 (obtained from the American Type Culture Collection, Manassas, Va.) was grown for 18 hours in trypticase soy broth (Becton, Dickinson and Company, Franklin Lakes, N.J.) at 37° C. The culture was diluted 1:100 in sterile, phosphate buffered saline (PBS) (Thermo Fisher Scientific Incorporated, Waltham, Mass.) using a 15 mL conical centrifuge tube.

Three circular punches (10 mm in diameter) of the Foam Article of Example 1 were prepared from regions of the foam that contained embedded gel. With both release liners removed, 10 microliter aliquots of the diluted S. aureus suspension were applied to each of the foam punches. Each aliquot was applied directly to the surface of the foam article containing the exposed gel. The inoculated punch samples were incubated for 5 minutes at room temperature and then placed into separate 15 mL conical centrifuge tubes containing 10 mL of Dey-Englay (D/E) neutralizing broth (Becton, Dickinson and Company, Franklin Lakes, N.J.). This procedure was repeated using three 10 mm circular punches of the Foam Article of Comparative Example A.

Each tube containing 5 mL of D/E broth and a foam punch sample was sonicated for one minute in a sonicating water bath (Branson model 2510 obtained from Emerson Electric Company, St. Louis, Mo.) and then vortexed at maximum speed with a VWR Mini Vortexer (VWR International, Radnor, Pa.) for one minute. The neutralizing broth was serially diluted (10-fold dilutions) and then plated (1 mL) onto a 3M PETRIFILM Aerobic Count plate (3M Corporation, Maplewood, Minn.). The plates were incubated for 24-48 hours at 37° C. At the end of the incubation period, colonies on each plate were counted by visual inspection.

The mean log (colony forming units/sample) recovered from the foam articles (n=3) was calculated by adjusting the observed plate count based on the number of dilutions. Statistical significance was determined with a Student's two-tailed, unpaired t-test where a P value of less than 0.05 was considered to signify a statistically significant difference. The results are reported in Table 2.

### Example 3. Planktonic Microbial Kill Assay (Pseudomonas aeruginosa)

The same procedure as reported in Example 2 was followed with the exception that Pseudomonas aeruginosa strain number 15442 (obtained from the American Type Culture Collection) was used instead of Staphylococcus aureus. The results are reported in Table 2.

TABLE 2
Counts of Bacteria Recovered from Foam Articles

| | Mean log (colony forming units/sample) | | |
|---|---|---|---|
| | with standard deviation (SD) | | |
| | Foam Article of | Foam Article of | |
| | Example 1 | Comparative Example A | t-test |
| S. aureus | 2.48 (SD = 1.35) | 5.82 (SD = 0.08) | 0.00 |
| P. aeruginosa | <1 (below the limit of | 5.18 (SD = 1.36) | 0.01 |
| | detection) | | |

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. An article, comprising:
a polymer foam having a first major surface and a second major surface; and
discrete domains of a therapeutic composition at least partially surrounded by the polymer foam;
wherein an exterior surface of each discrete domain substantially conforms to a portion of the first major surface of the polymer foam;
wherein the article comprises a first major surface and a second major surface; and
wherein a portion of the first major surface of the polymer foam and the exterior surfaces of discrete domains form the first major surface of the article.

2. The article of claim 1, wherein the article is a wound dressing.

3. The article of claim 1, wherein discrete domains form a pattern.

4. The article of claim 1, wherein at least at one plane that is parallel to the first major surface of the article and between the first and the second major surface of the article, the cross section of each discrete domain is greater than the cross section of the exterior surface of the discrete domain.

5. The article of claim 1, wherein the diameter of discrete domains is at least about 100 microns.

6. The article of claim 1, wherein the exterior surfaces of discrete domains cover between 1% and 99% of the first major surface of the article.

7. The article of claim 1, further comprising a liner on top of or adjacent to the first major surface of the article.

8. The article of claim 1, further comprising additional discrete domains of the therapeutic composition at least partially surrounded by the polymer foam.

9. The article of claim 8, wherein the exterior surface of each additional discrete domain substantially conforms to a portion of the second major surface of the polymer foam.

10. The article of claim 8, wherein a portion of the second major surface of the polymer foam and the exterior surfaces of additional discrete domains form the second major surface of the article.

11. The article of claim 1, further comprising a second liner on top of or adjacent to the second major surface of the article.

12. The article of claim 1, wherein the polymer foam comprises materials selected from the group consisting of polyurethane, polyvinyl acetate, polyvinylaleohol, polyethylene, and silicone.

13. The article of claim 1, wherein the therapeutic composition is retained in the discrete domains.

14. The article of claim 1, wherein the therapeutic composition comprises an active agent selected from the group consisting of antimicrobial agents, antibiotics, antioxidants, platelet-derived growth factor, vitamin A, vitamin C, vitamin E, corticosteroids, silver sulphadiazine, polymixin B sulphate, fusidic acids, pirfenedine, interferon, therapeutic oils, plant extracts, animal extracts, pharmaceuticals, vitamins, hormones, antioxidants, emu oil, aloe vera, lavender oil, rosehip oil silver sulphadiazme, polymixin B, fusidic acic and pirfenedine.

15. The article of claim 1, wherein the therapeutic composition comprises an antimicrobial agent.

16. A method of making a wound dressing, comprising:
depositing a therapeutic composition onto a surface of a liner to form a printed surface; and
disposing a foam material on the printed surface.

17. The method of claim 16, further comprising curing the foam material to form a foam on the printed surface.

18. The method of claim 16, wherein depositing the therapeutic composition comprises depositing the therapeutic composition in a pattern onto the liner.

19. The method of claim 16, further comprising removing the liner from the foam.

20. A method of making a wound dressing, comprising:
depositing a first therapeutic composition onto a surface of a first liner to form a first printed surface;
depositing a second therapeutic composition onto a surface of a second liner to form a second printed surface; and
disposing a foam material between the first printed surface and the second printed surface.
